(19) United States
(12) Patent Application Publication
(10) Pub. No.: US 2022/0296330 A1
(43) Pub. Date: Sep. 22, 2022

(54) SHIPPING AND SUPPLY SYSTEM FOR MEDICAL SHARPS

(71) Applicant: ULTIMED, INC., Excelsior, MN (US)

(72) Inventors: James Erickson, Mound, MN (US); Thomas P. Sauro, Rosemount, MN (US)

(21) Appl. No.: 17/698,843

(22) Filed: Mar. 18, 2022

Related U.S. Application Data
(60) Provisional application No. 63/163,298, filed on Mar. 19, 2021.

(51) Int. Cl. A61B 50/36; B65D 77/04

(52) U.S. Cl. CPC A61B 50/362; B65D 77/04; A61B 2050/0066

(57) ABSTRACT

The disclosure relates to an assembly for storing, dispensing, disposing, and shipping medical products such as sharps or pharmaceuticals. The assembly includes an outer container, an inner container, a unit configured for storing and dispensing unused medical products such as sharps or pharmaceuticals, and for receiving used medical sharps or unused pharmaceuticals. The inner container is adapted to receive the unit, and the outer container is adapted to receive the inner container.

### CROSS-REFERENCE TO RELATED APPLICATIONS

This application claims the benefit of priority under 35 U.S.C. § 119 of U.S. Provisional Application No. 63/163,298 filed Mar. 19, 2021, the entire disclosure of which is hereby incorporated by reference.

### TECHNICAL FIELD

This disclosure relates generally to the distribution of unused medical sharps and the consolidation and return of used medical sharps.

### BACKGROUND

Because of well-known health issues, the safe disposal of syringes and other “sharps” has long been a high priority for medical related professional facilities and industries. An increasing number of individuals are using medical sharps in private and public settings which use potentially creates a source of used and contaminated sharps outside of a relatively well controlled environment with established disposal protocols and collection points.

Medical delivery pens (hereinafter sometimes “MDPs”) have, more recently, become widely used instead of or in addition to syringes, e.g., by diabetics, who frequently inject themselves several times a day with accurately measured, adjustable, pre-selected amounts of insulin or other medication. Medical delivery pens include a reservoir of medication and a distal end adapted to be attached (usually by thread means) to a pen needle assembly (PNA). The pen needle assemblies typically include a removable thin sterile seal covering the proximal (large diameter) end of the said outer shield and a removable tube-like shield covering the distal portion of the hollow needle. The assembled pen needle assembly is then factory sterilized. The user of a pen needle assembly removes the seal from the outer shield, screws the pen into the proximal end of the pen needle housing, removes the outer and tube-like shields, sets the medical delivery pen for the desired dose of medication, and then inserts the distal end of the pen needle into the target tissue following which the medical delivery pen is actuated to deliver the desired dose of medication through the hollow needle into said tissue.

Many diabetics routinely administer medication to themselves several times a day by injection of a pre-selected quantity of insulin (or substitute medication) in liquid form; the correct amount of medication can be determined from prior professional medical instruction or by use of convenient portable blood analysis kits which are small, compact and provide rapid indicators of the user's blood sugar level. The several daily injections are often done away from the diabetic's home or residence which has made the use of the portable, convenient medical delivery pens widespread. The aforesaid testing kits and the medical delivery pens are relatively small in size and can easily fit within a woman's purse or equivalent. A typical scenario for a diabetic at a restaurant for a meal is to first use the blood sugar testing kit to obtain an indicator of his or her blood sugar level. This information then facilitates programming or adjusting the medical delivery pen to deliver the desired quantity of medication. Then the pen with an attached PN (a PNA without the outer protective shield) is used to inject the medication. These steps require a relatively short length of time and can be done with minimum loss of privacy.

MDPs are also widely used by doctors, nurses and other professionals in their duties. Many individuals will request (sometimes insist) that an injection be done with a pen needle rather than a syringe.

Devices which dispense unused pen needle assemblies and subsequently receive and store used pen needle assemblies are now available and are well accepted for convenience and safety in minimizing user contact with used medical sharps; however the used sharps must still be treated with caution until they are returned to an appropriate medical waste facility. Larger facilities such as hospitals and clinics typically have regularly scheduled services which collect containers of medical waste and used medical sharps. In areas where such routine collection of medical waste is not readily available, such as the user's home, it would be desirable to provide a system and method of providing a container of unused medical sharps with packaging for return to an appropriate medical waste treatment facility. Often this may be conveniently accomplished at a facility such as a pharmacy which dispenses the medical sharps.

### SUMMARY

This disclosure pertains to an assembly for distributing, storing, dispensing, disposing, and returning medical sharps.

An example assembly for storing, dispensing, disposing, and shipping medical products comprises an outer container, an inner container, and a unit containing unused medical products, wherein the inner container is adapted to receive the unit, wherein the outer container is adapted to receive the inner container.

Alternatively or additionally to any of the embodiments above, the unused medical products are unused medical sharps.

Alternatively or additionally to any of the embodiments above, the unit is configured for dispensing the unused medical sharps, the unit further configured for receiving used medical sharps.

Alternatively or additionally to any of the embodiments above, the medical sharps are pen needles or hypodermic needles.

Alternatively or additionally to any of the embodiments above, the unit further comprises a dispensing port capable of providing access to a first compartment configured to receive unused medical sharps.

Alternatively or additionally to any of the embodiments above, the dispensing port is not capable of allowing used medical sharps to be inserted into the first compartment to which the dispensing port provides access.

Alternatively or additionally to any of the embodiments above, the unit further comprises a receiving port capable of providing access to a second compartment for receiving used medical sharps such that used medical sharps may be inserted into the second compartment.

Alternatively or additionally to any of the embodiments above, the unit further comprises a partition separating the first and second compartments.

Alternatively or additionally to any of the embodiments above, the outer container is configured to ship medical waste.

Alternatively or additionally to any of the embodiments above, the outer container includes a postage paid shipping label addressed to a medical waste disposal or collection facility.

Alternatively or additionally to any of the embodiments above, the assembly further comprises a sleeve configured to receive the outer container.

Alternatively or additionally to any of the embodiments above, the assembly further comprises a receptacle configured for receiving used medical sharps.

Alternatively or additionally to any of the embodiments above, the assembly further comprises at least one sealing element configured to seal the outer container for shipping.

Alternatively or additionally to any of the embodiments above, the assembly further comprises a liquid barrier configured to receive the unit, wherein the inner container is adapted to receive the unit inside the liquid barrier.

Alternatively or additionally to any of the embodiments above, the unused medical products include pharmaceuticals.

Alternatively or additionally to any of the embodiments above, the unused medical products are prefilled syringes containing the pharmaceuticals.

Alternatively or additionally to any of the embodiments above, the unused medical products are selected from the group consisting of pills, capsules, drug-eluting patches, inhalers, bottles, and pen injectors.

Another example assembly for storing, dispensing, and returning medical sharps comprises an outer container configured to ship used medical sharps, an inner container, a unit containing unused medical sharps, the unit configured to dispense the unused medical sharps and configured for receiving the medical sharps after use, and a sealable liquid barrier configured to receive the unit, wherein the inner container is adapted to receive the unit, wherein the outer container is adapted to receive the inner container.

Alternatively or additionally to any of the embodiments above, the outer container includes a postage paid shipping label addressed to a medical waste disposal or collection facility.

Alternatively or additionally to any of the embodiments above, the unused medical sharps are pen needles.

Alternatively or additionally to any of the embodiments above, the unused medical sharps are hypodermic needles.

Alternatively or additionally to any of the embodiments above, the assembly further comprises at least one sealing element configured to seal the outer container for shipping.

Alternatively or additionally to any of the embodiments above, the assembly further comprises a sleeve, wherein the sleeve is adapted to receive the outer container.

Alternatively or additionally to any of the embodiments above, the unit further comprises a dispensing port capable of providing access to a first compartment containing the unused medical sharps.

Alternatively or additionally to any of the embodiments above, the dispensing port is not capable of allowing used medical sharps to be inserted into the first compartment to which the dispensing port provides access.

Alternatively or additionally to any of the embodiments above, the unit further comprises a receiving port capable of providing access to a second compartment for receiving the used medical sharps such that the used medical sharps may be inserted into the second compartment.

Another example assembly for storing, dispensing, and disposing medical products comprises a unit containing unused medical products, a receptacle configured for receiving used and/or excess medical products, and a joining member joining the unit and receptacle.

An example method of distributing unused medical sharps and collecting used medical sharps comprises providing a unit for storing, dispensing, and disposing medical sharps, the unit containing a volume of medical sharps, providing a sealable liquid barrier configured to receive the unit, providing an inner container configured to receive the unit within the sealable liquid barrier, providing an outer container configured to receive the inner container, providing a sleeve configured to receive the outer container, inserting the unit into the liquid barrier, inserting the liquid barrier into the inner container, inserting the inner container into the outer container, and inserting the outer container into the sleeve to define a package, distributing the package to a user, separating the sleeve, outer container, inner container, liquid barrier, and unit, dispensing unused medical sharps from the unit, inserting used medical sharps into the unit, sealing the unit within the liquid barrier, inserting the liquid barrier into the inner container, then inserting the inner container into the outer container and sealing the outer container and shipping the sealed outer container containing used medical sharps to a facility for destruction of the used medical sharps.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF DRAWINGS

The disclosure may be more completely understood in consideration of the following detailed description of various embodiments in connection with the accompanying drawings, in which:

FIG. 1 is schematic view of one embodiment of an assembly for shipping and storing a sharps container configured for storing, dispensing, and disposing medical sharps;

FIG. 2 is a top view of the elements of FIG. 2 in assembled configuration;

FIG. 3 is a perspective view of an example sharps container;

FIG. 4 is a top view of the elements of FIG. 2 in an alternative assembled configuration;

FIG. 5 is a top view of the elements of another assembly embodiment; and

FIG. 6 is a top view of the elements of a further assembly embodiment.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, “an example”, “some examples”, “other examples”, etc., indicate that the embodiment(s) and example(s) described may include a particular feature, structure, or characteristic, but every embodiment or example may not necessarily include the particular feature, structure, or characteristic. Moreover, such phrases are not necessarily referring to the same embodiment or example. Further, when a particular feature, structure, or characteristic is described in connection with an embodiment or example, it would be within the knowledge of one skilled in the art to effect the particular feature, structure, or characteristic in connection with other embodiments or examples, whether or not explicitly described, unless clearly stated to the contrary. That is, the various individual elements described below, even if not explicitly shown in a particular combination, are nevertheless contemplated as being combinable or arrangeable with each other to form other additional embodiments or examples, or to complement and/or enrich the described embodiment(s) or example(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

A variety of units for dispensing unused medical sharps such as pen needles and/or pen needle assemblies, hypodermic needles, and the like as well as receiving and storing those medical sharps following use have been described in the art. These devices are distinguished in that they are capable of storing a quantity of medical sharps before and after use; dispensing unused sharps singly or in small quantities; and receiving as well as storing used sharps following use. Generally these dual purpose units are portable so that a medical sharp may be available for use where and when needed and to limit the time that a used medical sharp is possibly exposed before being returned to protective storage. See U.S. Pat. Nos. 6,685,017; 6,923,318; 6,923,319; 7,556,149; 8,434,616; and 10,252,008, the disclosures of which are incorporated herein by reference, for nonlimiting examples of such devices.

Containers for storing and mailing used medical sharps are known, and may include a sharps container in a labeled mailing box. These containers may be available for purchase by a consumer at retail locations where the unused medical sharps, such as pen needles, are also sold. In other situations, the user may receive or acquire the containers from a health care professional. This system requires the consumer to purchase or receive two items and to dispose of whatever container the unused pen needles came in. A significant disadvantage of this system is the necessity for the consumer to receive or acquire the separate sharps container with mailing box. If the consumer forgets, or the retailer does not have them in stock when the consumer purchases the unused pen needles, the consumer must keep the used pen needles until a proper disposal container is found. This may lead to some of the used pen needles being disposed of improperly, for example in the user's residential garbage. The present disclosure is directed to an assembly that eliminates the need for the consumer to purchase or acquire two separate items, and ensures the consumer always has a proper container to store and dispose of the used medical sharps and packaging to mail the used medical sharps to a facility for properly disposing of the medical sharps. The single item assembly may reduce the amount of used medical sharps waste being disposed of improperly.

Although the following description will focus on an assembly with a unit containing pen needles and pen needle assemblies for clarity, it will be appreciated that the inventive concept also may be applied to other medical sharps such as hypodermic needles (with or without attached syringe bodies), lancets, micro-needles, drug-eluting patches (with or without micro-needles), infusion sets, and the like.

These units are often distributed through retail channels which sell individual devices loaded with a plurality of medical sharps, such as pen needle assemblies in the illustrative example to be discussed in greater detail below, to an individual user. It is, however, less common to provide a similarly convenient method to return the used sharps to a medical waste disposal or collection facility, and unfortunately a number of such units may find their way into municipal waste streams. While the units do provide protection against accidental sticks and/or contact with any associated bodily fluids, it would be desirable to provide a system which provides the individual user a convenient way to send a unit containing used sharps to a medical waste disposal or collection facility.

This disclosure provides an assembly which is suited to be sold to an individual user through normal retail outlet channels such as pharmacies. The assembly provides a unit preloaded with a plurality of unused medical sharps such as pen needle assemblies, along with packaging configured for the return shipping of the unit when it contains used medical sharps to a medical waste disposal or collection facility. A medial waste disposal facility is a facility that disposes of medical waste in accordance with all municipal, state, and federal guidelines, and a collection facility is one that may collect medical waste from various sources including clinics and private citizens, and then transports the medical waste to a separate disposal facility. In some examples, the unused medical sharps may contain a pharmaceutical, such as single use prefilled syringes. Prefilled syringes are becoming a more common delivery device for various pharmaceuticals including monoclonal antibodies, interferons, vaccines, anti-coagulants, and biotech drugs. Many of these prefilled syringes are prescribed for patient self-administration. The prefilled syringes provide a convenient delivery device for the user, but also require the user to properly dispose of the sharp after use.

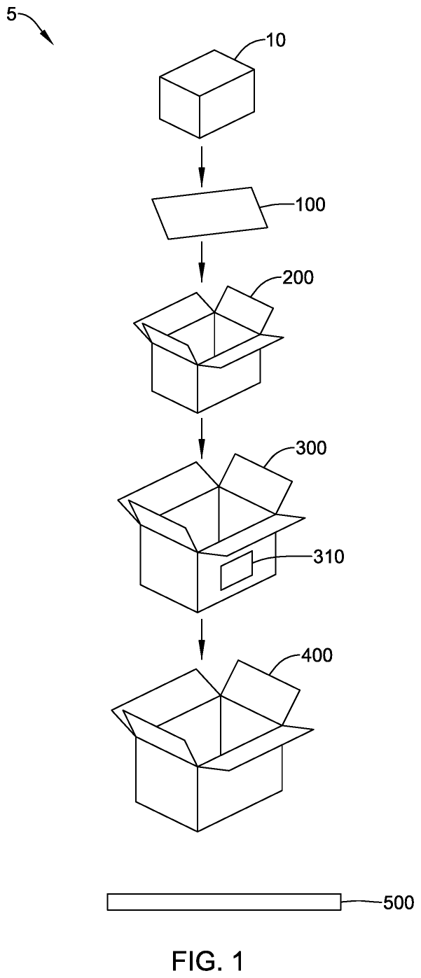

FIG. 1 illustrates an example assembly 5 including a unit 10, a sealable liquid barrier 100 configured to receive the unit 10, an inner container 200 configured to receive the unit 10 in the liquid barrier 100, an outer container 300 configured to receive the inner container 200, a sleeve 400 configured to receive the outer container 300, and a sealing member 500 configured to seal the outer container 300. The unit 10 may include unused sharps. The unused sharps may be empty or they may contain a pharmaceutical. In some examples the unit 10 may include separate regions for housing unused and used sharps. In other examples, the unit 10 may include separate containers for housing unused and used sharps. In such an example, the separate containers making up the unit may fit side by side in the inner container 200. In some examples, the sealable liquid barrier 100 may be a bag. The outer container 300 may have a pre-printed label 310 addressed to a medical waste disposal facility designated for handling the used medical sharps. In some embodiments, the inner container 200 may be eliminated, and the unit 10 in the sealable liquid barrier 100 may be placed directly into the outer container 300.

The sealable liquid barrier 100 may be a watertight polyethylene bag of at least 4 mil thickness and sized to receive the unit 10. The liquid barrier 100 may include a watertight sealing means, such as a zip-type seal. In other embodiments, a separate sealing member may be included, such as a twist tie or zip tie.

The inner container 200 and the outer container 300 may be boxes made of fiberboard, such as corrugated cardboard. The corrugated cardboard may be single walled, double walled, or triple walled. In some embodiments, the inner container 200 and/or the outer container 300 may include an inner coating of wax, polyurethane, lacquer or other material to provide a moisture barrier. The outer container 300 is made of 200 pound grade corrugated cardboard, as per United States Postal Service (USPS) requirements for shipping of sharps medical waste. In some embodiments, the inner container 200 may also be made of 200 pound grade corrugated cardboard.

The inner container 200 may be plain brown with no markings, while the outer container 300 may be white and may include a label 310 containing the applicable labeling information per the USPS requirements for shipping sharps medical waste, including biohazard labeling. The label 310 may be printed on the outer container 300 or may be fixedly attached such that it cannot be detached intact. The label 310 may include sufficient postage for mailing the outer container 300 with inner container 200 and unit 10 when it contains used sharps.

In some embodiments the inner container 200 and/or the outer container 300 may be at least partially made from recycled materials. In other embodiments, the inner container 200 and/or the outer container 300 may alternatively be made of polyethylene or extruded plastic, provided they meet the 200 pound crush standard required by the USPS. The sealing member 500 may be one or more pieces of packing tape. In some embodiments, the packing tape is water-resistant, and may be two inches wide. In some embodiments, the total length of the sealing member 500 piece(s) is sufficient to seal the joints of the outer container 300.

The sleeve 400 may be a thinner layer than the inner and outer containers, and may contain retail labeling. The sleeve 400 may contain all of the labeling and information customarily present on the retail package of the unused medical sharps. The sleeve 400 is not a structural element, as it encloses the outer container 300. In some embodiments, the sleeve 400 may be paper, cardstock, or cardboard and may cover all six sides of the outer container 300 or it may cover only four sides, leaving the ends of the outer container 300 visible. In other embodiments, the sleeve 400 may be a polymer wrapping, such as a plastic sleeve or shrink-wrap, and may be opaque such that the label 310 on the outer container 300 is not visible.

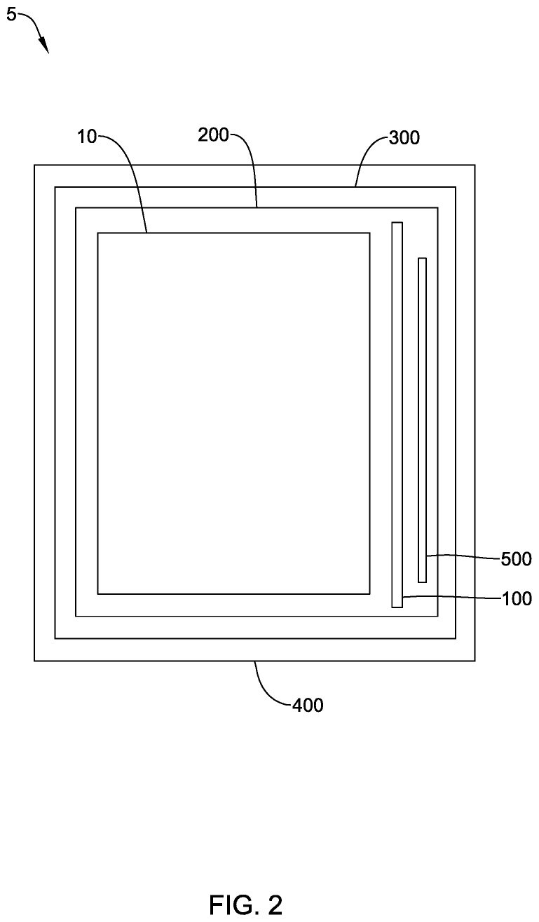

The inner container 200 is sized to receive the unit 10 in the liquid barrier 100 in a secure and snug fit to prevent breakage during shipping. The outer container 300 is sized to receive the inner container 200 is a secure, snug fit. The clearance between the inner container 200 and the outer container 300 may be about 1/16 inch to about ½ inch. In some embodiments, a desiccant may be added to the inner container 200 and/or the outer container 300. FIG. 2 is a top view illustration of the assembly 5 in the assembled configuration as it would be provided to the retailer, with the sleeve 400, outer container 300, and inner container 200 opened to show their contents. While the various elements are illustrated in FIG. 2 as being spaced apart, it will be understood that the unit 10, inner container 200, outer container 300, and sleeve 400 will be sized to fit closely within one another such that minimal shifting occurs during transportation. The nested configuration of the unit 10, inner container 200, and outer container 300 within the sleeve 400 saves space on the retail shelf and provides a convenient single-element purchase for the consumer.

The assembly 5 may contain a step-by-step instruction sheet that clearly details the proper sequence and method of assembling the unit 10 containing used medical sharps into the liquid barrier 100, inner container 200 and outer container 300 prior to mailing to prevent package failure during transport due to improper assembly.

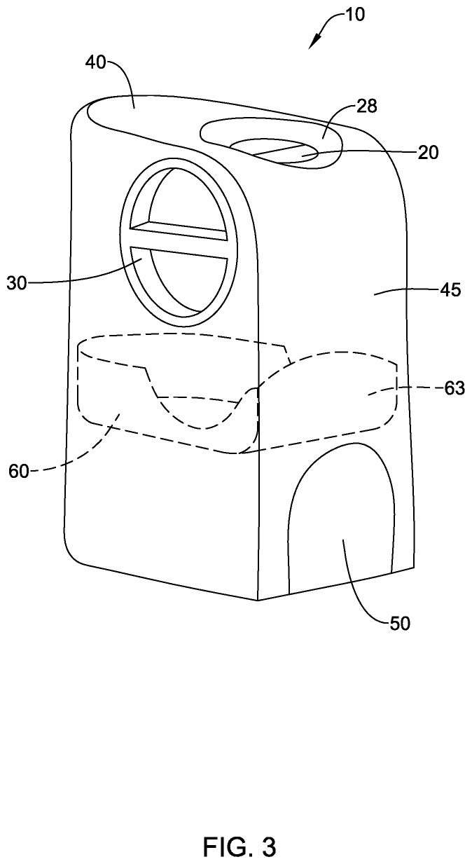

FIG. 3 illustrates a multi-functional unit 10 that provides a single apparatus for dispensing unused sharps and for the safe storage and disposal of used sharps. In some examples, the unused sharps may be prefilled with a pharmaceutical. The following description relates to pen needles as the sharps, however it will be understood that the unit 10 may be used to store, dispense, and collect any medical sharps. The unit 10 has a top 40, sides 45, and a bottom. Generally, the components of the unit 10 are locked, e.g., by adhesive and/or latches, to prevent opening the unit 10 or otherwise providing access to used pen needles contained therein. The unit 10 includes a receiving member 20 which assists the user in aligning and inserting the used pen needle and attached medical delivery pen into the receiving member 20. The unit 10 may also include a generally mating frustoconical segment 28 which provides a larger entry target.

Following removal of the pen needle from the medical delivery pen and removal of the medical delivery pen, the handle 30 may be rotated, clockwise or counterclockwise, to invert the attached receiving member 20 which allows the pen needle to fall or be ejected from the receiving member 20, with or without the assistance of an optional ejector mechanism (not shown). In addition to the features already discussed, FIG. 3 illustrates an interior partition 60, shown in phantom, which divides the unit into separate compartments for storing used pen needles and unused pen needle assemblies. In the unit illustrated, the lower compartment is configured for storing unused pen needle assemblies and door 50 opens to provide access to the storage compartment for unused pen needle assemblies. The partition 60 may serve as the bottom of the upper compartment which receives the used pen needles that are inserted through the receiving member 20. The partition 60 may be fixed in a single position or may move to alter the ratio of storage volume in the two compartments as unused needle assemblies are removed, used, and returned to the container with or without the associated pen needle cover. Partition 60 may also include a fluid absorbing layer 63 to minimize leakage of any fluids which may remain in or on the used pen needles.

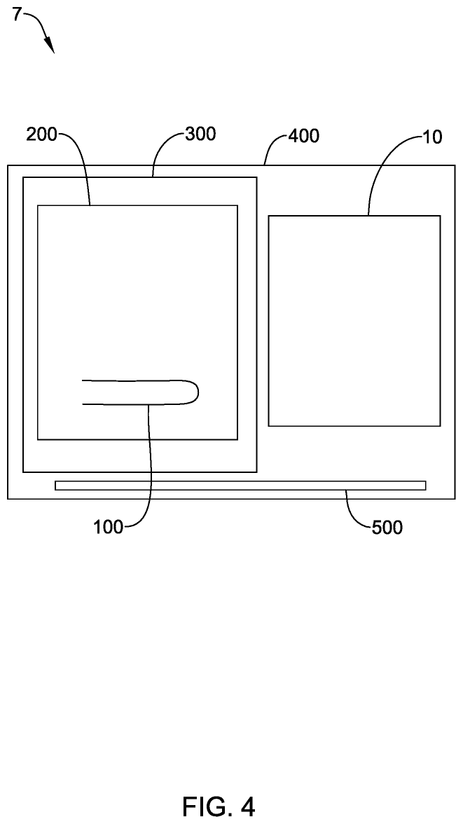

FIG. 4 is a top view illustration of an alternative assembly 7 in the assembled configuration as it would be provided to the retailer, with the sleeve 400, outer container 300, and inner container 200 opened to show their contents. The unit 10 containing unused medical sharps is positioned in the sleeve 400 separate from and next to the outer container 300 which contains the inner container 200 and liquid barrier 100. In some examples, the unit 10 may be a box of unused pen needles. The sealing member 500 may be disposed inside the sleeve 400 as shown, or in the outer container 300 or inner container 200. This configuration may allow the user to open the sleeve 400 and remove the unit 10 without having to open the outer container 300 and inner container 200.

The operation of the unit 10 is not influenced by the nature of the medical sharps which are to be dispensed and returned to the unit after use. Accordingly, the unit 10 may contain pen needle assemblies, hypodermic needles, syringes including hypodermic needles, lancets, and the like as well as mixtures thereof. Although the description of the unit 10 provided above has focused upon dual purpose devices which provide both the dispensing and the receiving functions in a single housing, it will be appreciated that the unit 10, as used herein, may also include separable components for the dispensing and receiving functions if desired. For example, the supply of unused medical sharps may be contained within a housing during the distribution phase and may be removed as a separate element of the unit by the end user who then uses the housing as the receiving element which will be returned to the container for consolidation and disposal.

The system of the disclosure may be used in the following manner. An empty unit 10 may be filled with a volume of medical sharps. The filled unit 10 and a sealable liquid barrier 100 may be disposed in an inner container 200 which is then placed (unsealed) in an outer container 300. The unsealed outer container 300 and one or more sealing member 500 are then placed into a sleeve 400 and the sleeve is sealed to form a package. The package is distributed to a retail location such as a pharmacy. A consumer purchases or otherwise acquires the assembly 5 and removes and discards the sleeve 400. The consumer separates and sets aside the outer container 300, sealing member 500, inner container 200, and liquid barrier 100. The consumer dispenses the pen needles or other sharps from the unit 10, and returns the used pen needles to the unit 10, as discussed above. When all of the pen needles have been used and returned to the unit 10, the user places the unit 10 into the liquid barrier 100 and seals the liquid barrier. The unit and liquid barrier assembly is then inserted into the inner container 200, which may be sealed or remain unsealed. The inner container 200 is then sealed and placed into the outer container 300 which is sealed. Both containers may be sealed with the sealing member(s) 500. The user then mails the outer container 300 using the USPS. The assembly 5 provides the user with a supply of pen needles in a convenient dispensing and return container and all of the packaging needed to return the used pen needles to a medical waste facility. The only element to be disposed of by the user is the cardboard sleeve 400, which may be placed in the user's residential recycling or garbage bin.

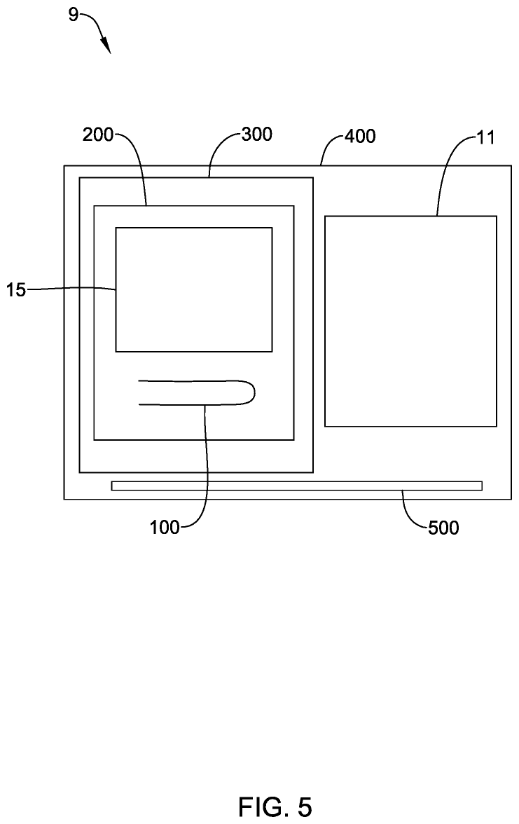

FIG. 5 is a top view illustration of another assembly 9 in the assembled configuration as it would be provided to the retailer, with the sleeve 400, outer container 300, and inner container 200 opened to show their contents. In this embodiment, the unit 11 contains unused medical sharps but is not configured to receive the used medical sharps. A separate receptacle 15 configured for receiving the used medical sharps is provided, and may be disposed inside the inner container 200 for shipping. The unit 11 may be positioned in the sleeve 400 separate from and next to the outer container 300 which contains the inner container 200, receptacle 15, and liquid barrier 100. In other examples the unit 11 and the receptacle 15 may both be disposed inside the inner container 200, in a side-by-side configuration. In some examples, the unit 11 may be a box of unused pen needles. The sealing member 500 may be disposed inside the sleeve 400 as shown, or in the outer container 300 or inner container 200. This configuration provides a unit 11 containing unused medical sharps along with the receptacle 15, sealable liquid barrier 100, inner container 200, and outer labeled shipping container 300 together in a single sleeve 400. This example provides the user with a single item to purchase or acquire that includes the unused medical sharps and all the materials and containers needed to safely dispose of the used medical sharps, but without requiring a special unit configured for both dispensing and receiving the medical sharps. The unit 11 may be a conventional cardboard box filled with unused medical sharps, and the receptacle 15 may be a conventional medical sharps container. Assembly 9 would be acquired by the user and sleeve 400 opened and discarded. The sharps in unit 11 would be used and placed into the receptacle 15. Once all sharps are used and placed in the receptacle 15, the unit 11 may be discarded. The receptacle 15 would be sealed inside the liquid barrier 100, which would then be placed in the inner container 200 which would then be placed in the outer container 300, sealed with sealing member 500, and placed in the mail. The outer container 300 may contain all labeling and postage required to ship the filled outer container 300 to a suitable medical waste disposal facility.

In another embodiment, the sleeve 400 may be replaced with shrink-wrap, a band, or one or more glue spots (not shown). The band may be paper, cardboard, or a flexible member. In the embodiment illustrated in FIG. 2, the sleeve 400 may be eliminated and the outer container 300 may have shrink-wrap or a band containing labeling and instructions disposed on the outside of the outer container 300. In the embodiments illustrated in FIGS. 4 and 5, the sleeve 400 may be eliminated and unit 10 or 11 may be shrink-wrapped, banded, or glued to the outer container 300, with the sealing member 500 disposed inside the outer container 300 or inner container 200.

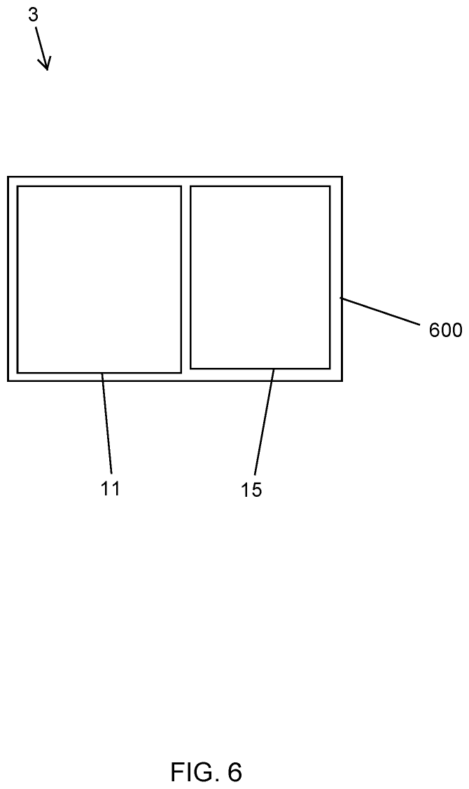

FIG. 6 is a top view illustration of a further assembly 3 in the assembled configuration as it would be provided to the retailer. In assembly 3, the unit 11 containing unused medical sharps, such as pen needles, and the receptacle 15 for receiving used medical sharps may be bundled together without the outer container 300, inner container 200, liquid barrier 100, and sealing member 500. As shown in FIG. 6, the assembly 3 includes only the unit 11 containing unused medical sharps, and the receptacle 15 for receiving used medical sharps, bundled together by a joining member 600. The joining member 600 may be a sleeve 400 as described above. In other embodiments, the joining member 600 may include shrink-wrap, a paper, cardboard or flexible band, one or more glue spots, or any other element configured for joining the unit 11 and receptacle 15. The joining member 600 may permanently join the unit 11 and receptacle 15 (glue spots) or it may be a removable joining member configured to be removed by the user (sleeve, shrink-wrap, band).

In addition to storing, dispensing, disposing, and shipping medical sharps as discussed above, the assemblies 5 may also be configured for dispensing and disposing of unused or excess pharmaceuticals, with or without a sharps element. These assemblies are also suited to be sold to an individual user through normal retail outlet channels such as pharmacies. In such an assembly, the unit 10 may be preloaded with a supply of pharmaceuticals. The pharmaceuticals may or may not include sharps. The pharmaceuticals may be provided in any suitable form or container, including prefilled syringes (with or without needles), pen injectors, any other type of sharps, tablets, capsules, powder, drug-eluting patches, liquid in bottles or vials, inhalers, etc. In addition to the unit 10 containing the pharmaceuticals, the assembly may include the packaging described in assembly 5 above, such as the sealable liquid barrier 100 configured to receive the unit 10, the inner container 200 configured to receive the unit 10 in the liquid barrier 100, the outer container 300 configured to receive the inner container 200, the sleeve 400 configured to receive the outer container 300, and the sealing member 500 configured to seal the outer container 300, as described above. The inclusion of the return packaging and label allows the user to easily return any used, unused or excess pharmaceuticals to a medical waste disposal facility. This may prevent the improper disposal of pharmaceuticals into residential waste treatment facilities.

Various modifications and alterations of this invention will become apparent to those skilled in the art without departing from the scope and principles of this invention, and it should be understood that this invention is not to be unduly limited to the illustrative embodiments set forth hereinabove. Changes may be made in details, particularly in matters of shape, size, and arrangement of steps without exceeding the scope of the disclosure. This may include, to the extent that it is appropriate, the use of any of the features of one example embodiment being used in other embodiments. All publications and patents are herein incorporated by reference to the same extent as if each individual publication or patent was specifically and individually indicated to be incorporated by reference.

What is claimed is:

1. An assembly for storing, dispensing, disposing, and shipping medical products comprising:
an outer container;
an inner container; and
a unit containing unused medical products;
wherein the inner container is adapted to receive the unit;
wherein the outer container is adapted to receive the inner container.

2. The assembly of claim 1, wherein the unused medical products are unused medical sharps.

3. The assembly of claim 2, wherein the unit is configured for dispensing the unused medical sharps, the unit further configured for receiving used medical sharps.

4. The assembly of claim 2, wherein the unused medical sharps are pen needles or hypodermic needles.

5. The assembly of claim 3, wherein the unit further comprises a dispensing port capable of providing access to a first compartment configured to receive the unused medical sharps.

6. The assembly of claim 5, wherein the dispensing port is not capable of allowing used medical sharps to be inserted into the first compartment to which the dispensing port provides access.

7. The assembly of claim 6, wherein the unit further comprises a receiving port capable of providing access to a second compartment for receiving used medical sharps such that used medical sharps may be inserted into the second compartment.

8. The assembly of claim 7, wherein the unit further comprises a partition separating the first and second compartments.

9. The assembly of claim 1, wherein the outer container is configured to ship medical waste.

10. The assembly of claim 9, wherein the outer container includes a postage paid shipping label addressed to a medical waste disposal or collection facility.

11. The assembly of claim 10, further comprising a sleeve configured to receive the outer container.

12. The assembly of claim 2, further comprising a receptacle configured for receiving used medical sharps.

13. The assembly of claim 1, further comprising a liquid barrier configured to receive the unit, wherein the inner container is adapted to receive the unit inside the liquid barrier.

14. The assembly of claim 1, wherein the unused medical products include pharmaceuticals.

15. The assembly of claim 14, wherein the unused medical products are prefilled syringes containing the pharmaceuticals.

16. The assembly of claim 14, wherein the unused medical products are selected from the group consisting of pills, capsules, drug-eluting patches, inhalers, bottles, and pen injectors.

17. An assembly for storing, dispensing, and returning medical sharps comprising:
an outer container configured to ship used medical sharps;
an inner container;
a unit containing unused medical sharps, the unit configured to dispense the unused medical sharps and configured for receiving unused the medical sharps after use; and
a sealable liquid barrier configured to receive the unit;
wherein the inner container is adapted to receive the unit;
wherein the outer container is adapted to receive the inner container.

18. The assembly of claim 17, wherein the outer container includes a postage paid shipping label addressed to a medical waste disposal or collection facility.

19. The assembly of claim 17, wherein the unit further comprises a dispensing port capable of providing access to a first compartment containing the unused medical sharps, wherein the dispensing port is not capable of allowing used medical sharps to be inserted into the first compartment to which the dispensing port provides access, wherein the unit further comprises a receiving port capable of providing access to a second compartment for receiving the used medical sharps such that the used medical sharps may be inserted into the second compartment.

20. A method of distributing unused medical sharps and collecting used medical sharps comprising:
providing a unit for storing, dispensing, and disposing medical sharps, the unit containing a volume of medical sharps;
providing a sealable liquid barrier configured to receive the unit;
providing an inner container configured to receive the unit within the sealable liquid barrier;
providing an outer container configured to receive the inner container;
providing a sleeve configured to receive the outer container;
inserting the unit into the liquid barrier, inserting the liquid barrier into the inner container, inserting the inner container into the outer container, and inserting the outer container into the sleeve to define a package;
distributing the package to a user;
separating the sleeve, outer container, inner container, liquid barrier, and unit;
dispensing unused medical sharps from the unit;
inserting used medical sharps into the unit;
sealing the unit within the liquid barrier;
inserting the liquid barrier into the inner container, then inserting the inner container into the outer container and sealing the outer container; and
shipping the sealed outer container containing used medical sharps to a facility for destruction of the used medical sharps.
